(19) United States
(12) Patent Application Publication
(10) Pub. No.: US 2007/0269512 A1
(43) Pub. Date: Nov. 22, 2007

(54) GASTRORETENTIVE SUSTAINED RELEASE FORMULATIONS

(76) Inventors: Wenhua W. Wang, Newtown, PA (US); John J. Ryan IV, Jamison, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/751,159

(22) Filed: May 21, 2007

Related U.S. Application Data
(60) Provisional application No. 60/802,539, filed on May 22, 2006.
(60) Provisional application No. 60/815,115, filed on Jun. 20, 2006.

(51) Int. Cl. A61K 9/22; A61K 31/4965

(52) U.S. Cl. 424/468; 514/255.06

(57) ABSTRACT

The present invention is directed to a gastroretentive sustained release formulation, processes for preparing said gastroretentive sustained release formulation and the use thereof.

### CROSS REFERENCE TO RELATED APPLICATIONS

This present application claims benefit of U.S. Provisional Patent Application Ser. No. 60/802,539, filed May 22, 2006, and U.S. Provisional Patent Application Ser. No. 60/815,115, filed Jun. 20, 2006, which is incorporated herein by reference in its entirety and for all purposes.

### FIELD OF INVENTION

The present invention relates to a pharmaceutical composition containing a therapeutic agent that is retained in the stomach or upper part of gastrointestinal tract for controlled delivery of medicament for improved local treatment and improved absorption in the upper part of the gastrointestinal tract for effective therapeutic results. Furthermore, the present invention also provides a method for preparation of said dosage form and a method of treatment by administering said dosage form.

### BACKGROUND OF THE INVENTION

Oral administration of a drug is perhaps the least predictable route of drug administration, yet it is the route that is used most frequently. Some aspects of unpredictability include a wide range of highly variable conditions such as pH, agitation intensity and composition of the gastrointestinal (GI) fluids as a dosage form passes down the GI tract. One particular aspect of the unpredictability is in controlling the site of absorption throughout the tract. Certain therapeutic agents are only absorbed by certain portions of the GI tract, such as in the stomach or upper portion of the tract.

The absorption of a therapeutic agent by the GI tract is dictated by the location of the agent in the gastrointestinal tract as well as by the GI contents. Some agents are more efficiently absorbed from the upper part of the tract while others are absorbed from the lower parts of the tract. Therefore, in instances where the drug is not absorbed uniformly over the length of the GI tract, the rate of absorption may not be constant in spite of the drug delivery system delivering the agent at a constant rate into the GI fluids.

Oral medications are also generally administered as immediate release dosage forms. The major disadvantage of such immediate release preparations is the repeated frequency of drug administration and fluctuations in drug plasma levels. Use of an oral controlled release preparation circumvents these problems. Various researchers have attempted to design controlled release, oral drug delivery systems that overcome these problems and deliver a therapeutic agent at a constant rate as the agent passes down the length of the GI tract.

Controlled drug delivery systems are designed to deliver a therapeutic agent in such a way that the level of the agent is maintained within a particular therapeutic window such that effective and safe blood levels are maintained for a period as long as the system continues to deliver the drug at a particular rate. Such controlled delivery usually results in substantially constant blood levels of the therapeutic agent as compared to fluctuations observed with immediate release dosage forms. Controlled delivery may also result in optimum therapy, reduce the frequency of dosing and may also reduce the severity of side effects.

There are numerous advantages associated with the use of controlled drug delivery systems. The main benefit in controlled drug therapy is the improvement in efficiency of the treatment. Controlled drug therapy reduces the required frequency of administration, and so single doses at periodic intervals are sufficient and may result in improved patient compliance.

A variety of controlled release dosage form designs have been reported in the literature. These controlled drug delivery systems are based on different modes of operation and have been variously named, for example, as dissolution controlled systems, diffusion controlled systems, ion exchange resins, osmotically controlled systems, erodable matrix systems, pH independent formulations, swelling controlled systems and the like.

An ideal controlled drug delivery system should deliver the drug at a constant rate as the therapeutic agent passes through the GI tract. However, in such cases where the agent has a particular absorption site in the GI tract (i.e. absorption window), as in the stomach or upper part of the small intestine for example, the agent may not be completely absorbed when administered in the form of a typical controlled drug delivery system.

PCT Application WO 85/04100 describes a composition for a gastrointestinal, pH-independent, sustained-release pharmaceutical unit dosage form comprising a non-compressed mixture of a therapeutic agent and from about 10 to about 60 percent by weight of a high molecular weight hydroxypropylmethylcellulose, and a method for production thereof in hard gelatin capsules.

U.S. Pat. No. 3,870,790 describes a solid, compressed buccal product containing low molecular weight hydroxypropylmethyl cellulose which has been modified by humidification and airdrying.

U.S. Pat. No. 4,226,849 describes tablets, lozenges, suppositories and/or other compressed dosage forms which have prolonged-release wherein hydroxypropylmethylcellulose has been subjected to hydrolysis and oxidation to generate a desired minimum concentration of carbonyl and carboxyl groups. The hydroxypropylmethylcellulose used was of low molecular weight.

U.S. Pat. No. 4,389,393 describes the use of high molecular weight hydroxypropylmethylcelluloses in low concentrations for achieving sustained drug release action from compressed solid dosage forms. The hydroxypropylmethylcelluloses used have a molecular weight above 50,000, a methoxyl content of 16-24% and hydroxypropoxyl content of 4-32%.

U.S. Pat. No. 3,065,143, U.S. Pat. No. 3,870,790, U.S. Pat. No. 4,167,588, U.S. Pat. No. 4,389,393 and U.S. Pat. No. 4,226,849 each describe that compression forms a diffusion barrier layer on the surface of the oral dosage form to provide sustained action.

U.S. Pat. No. 4,126,672 describes a dosage form wherein hydroxypropylmethylcellulose provides sustained release in a buoyant capsule dosage form and wherein the density of the capsules may have to be adjusted by the use of a fatty material (such as hydroxypropylmethylcellulose) so that the capsules float in the gastric fluid.

PCT Application WO 00/38650 describes a composition for a pharmaceutical dosage form for prolonged release of an active agent from a multilayered dosage form having a highly swellable layer and a drug layer, the dosage form being adapted for retention in the stomach for a prolonged period. The dosage form upon contact with the aqueous fluid/gastric contents swells to a maximum extent leading to increased buoyancy of the dosage form and the whole dosage form will float on the surface of the gastric contents.

PCT Application WO 04/002445 describes a composition for a pharmaceutical dosage form containing an active ingredient which is retained in the stomach or upper part of the GI tract for controlled delivery of medicament for improved local treatment, and/or better absorption from the upper parts of the GI tract for effective therapeutic results, wherein said dosage form is preferably in the form of a bilayer tablet, in which one layer constitutes for spatial control and the other being for temporal control.

There thus remains a need for a gastroretentive, oral dosage form that effectively delivers a drug having an “absorption window” in the stomach or upper part of the small intestine such that the delivery is at a controlled rate over a period of time. In other words, there remains a need for alternative gastroretentive, sustained release formulations.

### SUMMARY OF THE INVENTION

The present invention is directed to the delivery of a therapeutic agent in the form of a gastroretentive, sustained release formulation.

An example of the present invention is the delivery of a therapeutic agent having an “absorption window” in the GI tract in the form of a gastroretentive, sustained release formulation.

Another example is the delivery of a therapeutic agent having an “absorption window” in the stomach or upper part of the small intestine in the form of a gastroretentive, sustained release formulation.

Another example is the delivery of a cardiovascular agent (such as a thrombin inhibitor) having an “absorption window” in the stomach or upper part of the small intestine in the form of a gastroretentive, sustained release formulation.

The present invention is directed to the delivery of a therapeutic agent in the form of a gastroretentive, sustained release monolayered formulation.

An example of the present invention is the delivery of a therapeutic agent in the form of a gastroretentive, sustained release monolayered formulation, comprising a polymer selected from hydroxypropylmethylcellulose (HPMC), hydroxypropylcellulose (HPC) and polyvinylacrylate (PVA) or mixtures thereof.

The gastroretentive, sustained release monolayered formulation of the present invention is a dosage form that floats on the surface of the gastric contents, thus allowing controlled release of the therapeutic agent, such that the agent is delivered over a period of time, preferably over a period of 24 hours.

Furthermore, the formulation of the present invention releases the therapeutic agent having an absorption window in the stomach or upper part of the gastrointestinal tract in a slow, controlled manner for improved absorption and efficacy compared to other conventional and controlled release dosage forms.

As well, the formulation of the present invention provides a drug delivery system that can incorporate high and low dose medicament without compromising dosage form characteristics or properties with an acceptable size for oral administration.

### BRIEF DESCRIPTION OF THE FIGURES

FIG. 1: Dissolution rate of the tablets and capsules as prepared in Example 1.

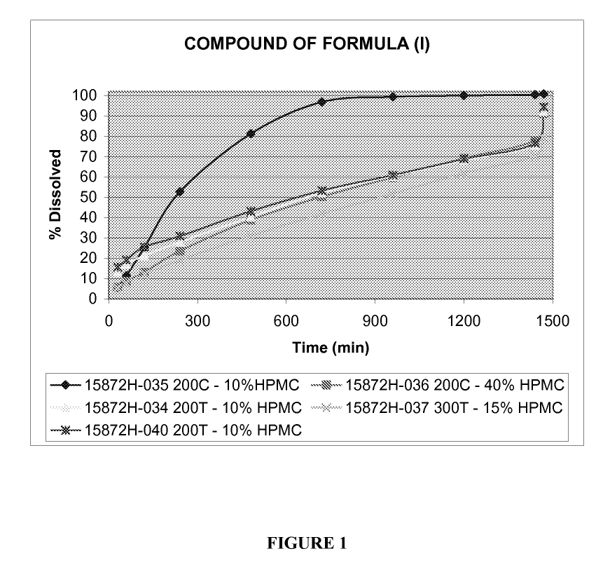

### DETAILED DESCRIPTION OF INVENTION

The present invention includes a pharmaceutical composition comprising an effective amount of the compound of formula (I) in a gastro-retentive, sustained release formulation.

The present invention relates to a pharmaceutical composition in the form of a buoyant, sustained release dosage form to prolong the delivery of the drug in the stomach or upper part of small intestine. Accordingly, the dosage form floats on the surface of the contents in the stomach, thus keeping the dosage form in the stomach where absorption can take place.

An example of the polymer used in the present invention includes, but is not limited to, a polymer selected from HPMC, HPC, PVA and the like or mixtures thereof.

For example, a pharmaceutically acceptable form for a gastroretentive, sustained release formulation of the compound of formula (I):

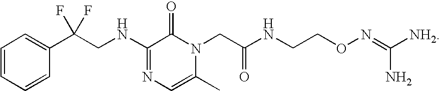

The present invention further includes the use of a process for making a composition or medicament comprising mixing the gastroretentive, sustained release formulation and an optional pharmaceutically acceptable carrier; and, includes those compositions or medicaments resulting from such a process. Contemplated processes include both conventional and unconventional pharmaceutical techniques.

Compositions or medicaments suitable for oral administration include solid forms such as slugs or capsules. Examples of the formulation of the present invention include optionally encapsulated slugs or capsules containing either a wet or dry powder or granulation optionally loosely compacted. The powder mixture of milled granulation can then be filled into capsules.

For preparing the compositions, the gastroretentive, sustained release formulation is mixed with a pharmaceutical excipient, e.g., conventional ingredients such as diluents, binders, adhesives, lubricants, antiadherents, glidants, antioxidants and colorants.

The final dosage form may also be coated with suitable coating materials for either functional or non-functional use known to those skilled in the art of formulation development without hindering the release of therapeutic agent from the gastroretentive dosage form.

Suitable fillers include fillers microcrystalline cellulose (i.e., AVICEL™ microcrystalline cellulose available from FMC Corp.), lactose, silicified MCC, mannitol, sorbitol and any other diluent that does not have disintegrant properties and the like.

Suitable glidants, lubricants and antiadherents include, but are not limited to, stearates (magnesium, calcium and sodium), stearic acid, talc waxes, Stearowet®, boric acid, sodium chloride, DL-leucine, carbowax 4000, carbowax 6000, sodium oleate, sodium benzoate, sodium acetate, sodium lauryl sulfate, magnesium lauryl sulfate, and the like. Preferred glidant is magnesium stearate.

Additionally, antioxidants such as butylated hydroxytoluene (BHT), ascorbic acid, sodium bisulfite and the like may be added. Preferred antioxidant according to the present invention is butylated hydroxytoluene. As well, colorants and coatings may be added or applied to the dosage form for ease of identification of the drug or for aesthetic purposes.

These excipients are formulated with the therapeutic agent to provide an accurate, appropriate effective amount of the therapeutic agent such that a therapeutically or prophylactically effective release profile results.

Generally, such excipients are mixed with the therapeutic agent to form a solid preformulation composition containing a homogeneous mixture of the therapeutic agent or a pharmaceutically acceptable salt thereof.

Said formulation may be formed by various common methods such as, for example, wet or dry granulation.

After wet granulation, the granulated material is dried and then ground. The resulting particulate is admixed with optionally present pharmaceutically acceptable excipients and then compressed into a slug containing from about 0.01 mg to about 500 mg of the therapeutic agent. The resulting slug may then be coated and fitted into capsules of an appropriate size.

Advantageously, the gastroretentive, sustained release formulation comprising the micronized form of the therapeutic agent may be administered in a single daily dose. A micronized form of the therapeutic agent includes those having a particle size of up to 300 microns.

Alternatively, the composition may be formulated such that the total daily dose may be administered in divided doses of two, three or four times daily. An example of the present invention is a gastroretentive, sustained release formulation in a form suitable for once-daily administration.

The composition or medicament may contain from about 0.001 mg to about 5000 mg (preferably, from about 50 to about 500 mg) of the therapeutic agent and may be constituted into any form suitable for the mode of administration selected for a subject in need. A contemplated effective amount may range from about 0.001 mg to about 20 mg/kg of body weight per day, or in the range of from about 0.1 to about 3 mg/kg of body weight per day. The composition or medicament may be administered according to a dosage regimen of from about 1 to about 5 times per day.

For oral administration, the composition or medicament is preferably in the form of a capsule containing, e.g., 2.5, 5.0, 10.0, 15.0, 25.0, 50.0, 100, 150, 200, 250 and 500 milligrams of the therapeutic agent for the symptomatic adjustment of the dosage to the patient to be treated.

Optimal dosages will vary depending on factors associated with the particular patient being treated (e.g., age, weight, diet and time of administration), the severity of the condition being treated, the compound being employed, the amount of compound dosed to achieve a non-toxic, therapeutic effect, the mode of administration and the strength of the preparation. The use of either daily administration or periodic dosing may be employed.

The polymers used in the formulation of the present invention can be various cellulosic derivatives either synthetic or semisynthetic, such as HPMC, HPC or PVA or mixtures thereof in combination with optionally present pharmaceutically acceptable excipients.

In an embodiment of the present invention the therapeutic agent is a compound of Formula (I).

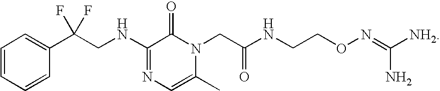

As an example, the compound of Formula (I) is substantially pure, preferably the compound of Formula (I) is free of salt form. The compound of Formula (I) may be present in an amount ranging from about 10 mg to about 400 mg per unit dose, preferably from about 150 mg to about 250 mg, more preferably in a dose of about 200 mg per unit dose.

As another example the compound of Formula (I) is present in a relative amount (w/w) of about 20% to about 60%, preferably from about 30% to about 50%, more preferably about 40%.

In an embodiment of the present invention, the HPMC polymer is Methocel (brand of HPMC).

In another embodiment, the grade of Methocel is KI00, having a viscosity of about 80-120 cps, a loss on drying (LOD) of less than 5.0%, a methoxyl content of about 19.0-24.0% and a hydroxypropoxyl content of about 7.0-12.0%. Other grades that may be used include: K4M, having a viscosity of about 3000-5600 cps, a LOD of less than 5.0% max, a methoxyl content of about 19.0-24.0% and a hydroxypropoxyl content of about 7.0-12.0%); K15M, having a viscosity of about 11,250-21,000 cps, a LOD of less than 5% max, a methoxyl content of about 19.0-24.0% and a hydroxypropoxyl content of about 7.0-12.0%); or, K100M, having a viscosity of about 80,000-120,000 cps, a LOD of less than 5% max, a methoxyl content of about 19.0-24.0% and a hydroxypropoxyl content of about 7.0-12.0%.

Another embodiment includes Methocel having a molecular weight in a range of between about 10,000 gm/Mol to about 2,000,000 gm/Mol, or in a range of between about 50,000 gm/Mol to about 500,000 gm/Mol, or in a range of about 100,000 gm/Mol.

In another embodiment, the grade of Methocel is K100LV Premium Controlled Release.

When HPMC is chosen as the polymer, the relative amount (w/w) ranges from about 5% to about 80%, interestingly from about 5% to about 50%, more interestingly from about 10% to about 80%, preferably from about 10% to about 40%.

In an embodiment of the present invention, the compression force is in a range of up to about 800 ft-lbs (i.e., 3559 Newtons).

In another embodiment of the present invention, the formulation includes a granulation wherein the agent is mixed with the polymer.

In another embodiment, the formulation includes a wet granulation which is then dried or a solid mixture powder or low compression weight slug in a capsule.

In an embodiment of the formulation of the present invention, microcrystalline cellulose is used as a filler. The relative amounts of microcrystalline cellulose ranges from 30% to about 60%, preferably the relative amount of microcrystalline cellulose is about 48.25%.

When relative amounts are mentioned, a person skilled in the art would be aware that the percentages should add up to 100% and that the other ingredients are taken in such an amount as to allow to add up to 100%.

The term “(w/w)” is well understood by the person skilled in the art as the mass of the specific ingredient divided by the total mass of the resulting mixture. Mostly relative amount of (w/w) are expressed as percentages.

According to the present invention, the therapeutic agent can belong to any drug class and be for use in any disorder by which the therapy or chemotherapy would be improved as a result of controlled drug delivery. The therapeutic agent may be pharmacologically or chemotherapeutically active itself, or may be converted into active species by a chemical or enzymatic process in the body.

A suitable therapeutic agent for use in the present invention are those from drug classes having an absorption-window and or site of action in stomach or in the upper part of the small intestine, including those therapeutic agents which do not show uniform absorption characteristics throughout the gastrointestinal tract.

Examples of other suitable therapeutic agents useful for treating, preventing or ameliorating a chronic or acute disease, disorder or condition include, but are not limited to, are cardiovascular agents (such as thrombin inhibitors), antibiotics, anti-cancers, anti-fungals, anti-fibrial and antiviral agents, lipid lowering agents, non-steroidal anti-inflammatory agents, anti-ulcer agents, drugs for respiratory therapy, dopaminergic agents, skeletal muscle relaxants, anti-epileptic, immunosupressants, anti-gout, antipsychotics and the like.

Illustrative examples of drugs that are suitable for the present invention include thrombin inhibitors such as the compound of formula (I) and others such as DPC-423, DPC-602, DPC-906 (razaxaban), GSK's 813893, Portola's Factor Xa benzoxazinone inhibitors (formerly Millenium), FXV673 (otamixaban), DU-176b, KFA-1982, BAY-59-7939 (rivaroxaban), DX-9065a, YM-150, LY-517717, Exanta, SSR-182289, LB-30057, LB-30870, BIBR-1048 and Merck's thrombin inhibitors (see Saiah E. and Soares C., Small molecule coagulation cascade inhibitors in the clinic, Current Topics in Medicinal Chemistry, 2005, 5, 1677-1695); antibacterial/anti-infective agents, such as ofloxacin, ciprofloxacin, cefuroxime, cefatrizine, cefpodoxime, clarithromycin, loracarbef, azithromycin, cefadroxil, cefixime, amoxycillin and the like; antivirals, such as acyclovir; cardiovascular agents, such as diltiazem, captopril, and the like; lipid lowering agents such as simvastatin, lovastatin, atorvastatin, and the like; non-steroidal anti-inflammatory agents such as etodolac, ketorolac, and the like; anti-ulcer agents, such as ranitidine, famotidine and the like; drugs for respiratory diseases, such as foxofenadine, pseudoephedrine, phenylpropanolamine, dextromethorphan, chlorpheniramine, and the like; dopaminergic agents, such as bromocriptine; immunosuppressants, such as cyclosporin; skeletal muscle relaxants, such as baclofen: anti-gout agents, such as allopurinol: and the like: antidiabetic agents such as acarbose, glipizide and the like.

In another embodiment of the invention the therapeutic agents are in a particular polymorphous form. Alternatively the therapeutic agent may be in an amorphous form. Certain therapeutic agents may be present in a particular solvate formed with water (i.e. hydrate) or common organic solvent (e.g. ethanolates).

The term “pharmaceutically acceptable” refers to molecular entities and compositions that are of sufficient purity and quality for use in the formulation of a composition of the present invention and that, when appropriately administered to an animal or a human, do not produce a toxic, adverse, allergic or other untoward reaction. Since both human use (prescriptive and over-the-counter) and veterinary use are equally included within the scope of the present invention, a pharmaceutically acceptable formulation would include a composition for either human or veterinary use.

The present invention is also directed to a method for use of a therapeutic agent in the gastroretentive sustained release formulation for treating, preventing or ameliorating a chronic or acute cardiovascular disease, disorder or condition in a subject in need thereof comprising administering to the subject an effective amount of the therapeutic agent.

The present invention also includes the use of the compound of formula (I) for the manufacture of a medicament for treating a chronic or acute disease, disorder or condition in a subject in need thereof.

The term “administering,” with respect to the method of the present invention, refers to a means for treating, ameliorating or preventing a disease, disorder or condition as described herein with the compound of formula (I) specifically disclosed or a form thereof, which would obviously be included within the scope of the invention albeit not specifically disclosed.

Such methods include prophylactically or therapeutically administering an effective amount of the compound of formula (I) or a form thereof at different times during the course of a therapy or concurrently in a combination form. Prophylactic administration can occur prior to the manifestation of symptoms characteristic of a disease, disorder or condition such that the disease, disorder or condition is prevented or, alternatively, delayed in its progression. The instant invention is therefore to be understood as embracing all such regimes of simultaneous or alternating treatment and the term “administering” is to be interpreted accordingly.

The term “subject” as used herein, refers to a patient, such as an animal, preferably a mammal, most preferably a human, who has been the object of treatment, observation or experiment and is at risk of (or susceptible to) developing a disease, disorder or condition or having such a disease, disorder or condition.

The term “effective amount” refers to that amount of the compound of formula (I) or a form thereof that elicits a non-toxic biological or medicinal response in a tissue system, animal or human, that is being sought by a researcher, veterinarian, medical doctor, or other clinician, which includes treating, preventing or ameliorating the symptoms of the disease, disorder or condition being treated.

The effective amount of the compound of formula (I) or a form thereof exemplified in such a method is from about 0.001 mg/kg/day to about 300 mg/kg/day.

The term “medicament” refers to a product for use in treating, preventing or ameliorating a chronic or acute disease, disorder or condition.

The methods of the present invention further include therapeutically administering an effective amount of the compound of formula (I) in the present formulation with one or more therapeutic agents at different times during the course of a therapy or concurrently in a combination therapy.

A combination therapy may advantageously facilitate the use of a reduced effective dose of the compound of formula (I) and/or the therapeutic agent than would be recommended for the treatment of a particular disease, disorder or condition. Therefore, it is contemplated that the compound of formula (I) can be used before, during or after treatment with a particular therapeutic agent.

The following non-limiting examples describe the illustrative pharmaceutical compositions of the present invention and the means of carrying out the invention to obtain a pharmaceutical dosage form of various active agents for oral controlled release.

### EXAMPLE 1

Preparation of the Formulations

Formulation 15872H-034 represents 200 mg tablets with 10% HPMC. Formulation 15872H-035 represents 200 mg capsules with 10% HPMC. Formulation 15872H-036 represents 200 mg capsules with 40% HPMC. Formulation 15872H-037 represents 300 mg tablets with 15% HPMC.

| | 15872H-034 | | 15872H-035 | | 15872H-036 | | 15872H-037 | |
|---|---|---|---|---|---|---|---|---|
| Components | mg/tablet | % w/w | mg/tablet | % w/w | mg/tablet | % w/w | mg/tablet | % w/w |
| Compound of | 200.00 | 40.00 | 200.00 | 40.00 | 200.00 | 40.00 | 300.00 | 42.86 |
| Formula (I), | | | | | | | | |
| Micronized | | | | | | | | |
| Microcrystalline | 241.25 | 48.25 | 241.25 | 48.25 | 91.25 | 18.25 | 282.75 | 40.39 |
| Cellulose (Avicel | | | | | | | | |
| PH102) | | | | | | | | |
| Hypromellose - | 50.00 | 10.00 | 50.00 | 10.00 | 200.00 | 40.00 | 105.00 | 15.00 |
| Methocel K100LV | | | | | | | | |
| Prem CR | | | | | | | | |
| Butylated | 3.75 | 0.75 | 3.75 | 0.75 | 3.75 | 0.75 | 5.25 | 0.75 |
| Hydroxytoluene | | | | | | | | |
| (BHT) | | | | | | | | |
| Magnesium | 5.00 | 1.00 | 5.00 | 1.00 | 5.00 | 1.00 | 7.00 | 1.00 |
| Stearate (Non- | | | | | | | | |
| bovine) | | | | | | | | |
| Purified Water | | | | | | | 7.00 | 1.00 |
| (removed during | | | | | | | | |
| drying) | | | | | | | | |
| Totals | 500.00 | 100.0 | 500.00 | 100.0 | 500.00 | 100.0 | 700.00 | 100.0 |

The manufacturing process used in the present invention for the floating sustained release capsules as well as sustained release tablets was as follows:
1. The drug substance, microcrystalline cellulose, hydroxypropylmethylcellulose, and butylated hydroxytoluene were dry blended in a high shear mixer;
2. The material was wet granulated in a high shear mixer with purified water;
3. The wet granules were screened and dried;
4. The dried granules were sized by means of a mill or screen;
5. The sized granulation was blended with magnesium stearate;
6. The final blend may be directly filled into capsules or used to form slugs;
7. The slugs are formed using low compression force and then filled into capsules.

As described in Step 1, the appropriate amounts of the compound of Formula (I), the hydroxypropylmethyl-cellulose (HPMC) K100, the microcrystalline cellulose (MCC), and the butylated hydroxytoluene (BHT) were added to a Black and Decker Handy Chopper Plus. Prior to addition, the BHT was screened through a 20 mesh screen. The components were dry blended for approximately 10 seconds and visually inspected for uniformity.

As described in Step 2, 20 ml of water was added over an approximate time period of 5 seconds and the granulations were mixed an additional 10 seconds.

As described in Step 3, the mixed granulation was visually inspected to ensure the uniform dispersion of water, then was emptied onto aluminum foil and placed in the Hotpack Convection Oven for drying at 50° C. for a suitable period of time, wherein the drying time for individual dosage forms and loss on drying was as follows:

| Formulation | Dry Time (min) | Final LOD (%) |
|---|---|---|
| 15872H-034 | 36 | 3.26 |
| 15872H-035 | | |
| 15872H-036 | 40 | 2.48 |
| 15872H-037 | 37 | 5.47 |

As described in Step 5, After drying, each granulation was hand blended with 1% magnesium stearate in a 250 ml amber glass jar for 1 minute.

As described in Step 7, the blends were compressed as follows:

| Formulation | Compression (lbs) | Hardness (kp) | Avg Weight (mg) |
|---|---|---|---|
| 15872H-034 | 1600 | 15.1 | 501.1 |
| 15872H-035 | 300 | 1.4 | 501.4 |
| 15872H-036 | 400 | 1.1 | 500.7 |
| 15872H-037 | 1100 | 15.0 | 699.4 |

10 Tabs/Caps were produced for each formulation. Four were submitted for dissolution testing (Example 1) and six were reserved for the dog study (Example 2).

Dissolution Results

Dissolution data is shown in Table A as Percent Dissolved [avg (range) % relative standard deviation].

Formulation 15872H-034 represents 200 mg tablets with 10% HPMC (group of n=1) at 1600 lbs compression force. Formulation 15872H-035 represents 200 mg capsules with 10% HPMC (group of n=2) at 300 lbs compression force. Formulation 15872H-036 represents 200 mg capsules with 40% HPMC (group of n=2) at 400 lbs compression force. Formulation 15872H-037 represents 300 mg tablets with 15% HPMC (group of n=1) at 1100 lbs compression force.

TABLE A
Dissolution Data

| Time | | | |
|---|---|---|---|
| (min) | 15872H-034 | 15872H-035 | 15872H-036 |
| 30 | 17 | 11 (8.7-12.8) 27.3 | 3 (3.2-3.8) 12.9 |
| 60 | 21 | 20 (17.6-23.0) 18.9 | 7 (6.6-8.1) 14.7 |
| 120 | 27 | 42 (38.2-45.8) 12.8 | 15 (13.6-16.7) 14.3 |
| 240 | 38 | 64 (59.5-68.4) 9.8 | 30 (27.6-32.7) 11.9 |
| 480 | 57 | 88 (85.8-89.8) 3.2 | 55 (51.7-57.7) 7.7 |
| 720 | 67 | 97 (99.0-95.3) 2.7 | 73 (69.3-75.8) 6.3 |
| 960 | 76 | 99 (100.1-99.7) 1.7 | 85 (81.4-89.5) 6.7 |
| 1200 | 82 | 100 (101.0-99.7) 1.0 | 93 (89.8-96.5) 5.1 |
| 1440 | 86 | 100 (100.9-100.0) 0.6 | 95 (91.8-99) 5.3 |
| 1470 | 99 | 102 (101.8-101.3) 0.3 | 101 (100-102.2) 1.6 |

When comparing capsule dissolution, the data shows typical correlations between n and release rate; the higher the concentration, the slower the rate of release.

The data also shows the typical effect of compression force on the formulation. As the compression force increases the rate of release decreases.

Compression force has an effect on location of the dosage form during dissolution testing. The capsules floated on the surface of the dissolution bath medium; the tablets sunk and were anchored to the glass bottom of the dissolution chamber.

Due to the observations noted from dissolution testing, a floatability study was performed. Two capsules containing the same formulation as 15872H-036 (40% K100LV) were tested. One capsule was hand filled with powder and the second capsule contained a slugged dosage form (800 lbs force). Both capsules were placed in a 100 ml beaker containing 80 ml of 0.1N HCl Solution and a 1.5 inch stirring bar. The beakers were placed on a stirrer plate and the disintegration of the capsules was recorded by means of photographs approximately every 30 minutes for 380 minutes. At the completion of the study, both capsules were floating on the surface of the disintegration medium.

After the feasibility manufacturing of capsules containing 20% K100LV formulation on a fully automated encapsulation machine, another floatability study was performed. A capsule was placed in a 100 ml beaker containing 80 ml of 0.1N HCl Solution and a 1.5 inch stirring bar. Photographs recorded the disintegration approximately every 30 minutes. The capsule disintegrated after approximately 280 minutes and remained on the surface of the disintegration medium the length of the study. The increase in disintegration when compared to the previous study is due to the decrease in HPMC in the capsule.

### EXAMPLE 2

Pharmacokinetic Study

Nineteen female beagle dogs exhibiting good general health were chosen for this study. Each overnight-fasted dog in group 1 received an intravenous bolus dose administration (1 mg/kg) of the compound of Formula (I). Following i.v. dose administration, blood samples (˜3 mL) were collected via jugular venipuncture, or other suitable site, into EDTA tubes and placed on wet ice, at predose (0.0), 0.25, 0.5, 1, 2, 4, 8, and 24 hours post dose. Each overnight-fasted dog in groups 2-5 received a single oral dose (200 mg) of compound of Formula (I). Following oral dose administration, blood samples (˜3 mL) were collected via jugular venipuncture, or other suitable site, into EDTA tubes and placed on wet ice, at predose (0.0), 0.25, 0.5, 1, 2, 4, 8, 24, 36, and 48 hours post dose. Plasma was harvested by centrifugation, each plasma sample are split into approximately equal parts before being frozen at −70° C. Food was returned after the 4 hour blood sample is collected. All samples were processed within two hours of collection. All samples are labeled with the study number, test article, dose, time interval, species, and animal identification number.

Sample Analysis

One set of plasma samples were analyzed for unchanged compound of Formula (I) concentrations using a liquid chromatographic-triple quadrupole mass spectrometric (LC-MS/MS) assay procedure. Any remaining plasma was retained for possible metabolite profiling. The other set of plasma samples was used for measurement of coagulation parameters.

Data Analysis

Pharmacokinetic analysis of the plasma concentrations of the compound of Formula (I) was performed to determine the concentration at zero time (C₀) by the extrapolation of drug concentration back to the time of dosing (IV only), the maximum plasma concentration (Cₘₐₓ), time to maximum plasma concentration (tₘₐₓ), the area under the plasma concentration versus time curve extrapolated to infinity (AUC_{0-∞}), terminal half-life (t_{1/2}), plasma clearance (CL/F) and the apparent volume of distribution at steady state (Vdₛₛ IV only) using the WinNonlin Version 3.1 (Pharsight, Palo Alto, Calif.) validated computer program. The absolute bioavailability (F) of the oral dose will be calculated using the validated computer program Microsoft® Excel 2000 (vers. 9.0.3821 SR-1, Microsoft Corporation, Redmond, Wash.) by comparing the individual oral AUC value to the individual i.v. AUC value for each animal.

Results

Compound X can be measured by means of a biomarker. A biomarker is a biological measurement that provides information concerning the effects of a drug. In this case, activated partial thromboplastin time (aPTT), corresponds to the plasma concentrations of Compound X. The targeted therapeutic range of aPTT is between 1.5 and 2.5.

Tables B and C indicate the following:

1. HPMC behaved as would be expected in the dosage forms. Dose dumping delivery of the drug as found in 15860H-001 (drug-filled capsules without HPMC) was not found in the dosage forms that contain HPMC (15872H-034, 035, 036).

2. Compound X had very poor colonic absorption. Comparing the aPTT values between the capsules and tablets indicate that the capsules stayed in the upper GI tract for a longer period of time. Both sustained release floating capsule forms (15872H-035 and 036) when compared to the 200 mg capsule which did not contain HPMC and the 200 mg tablet (15860H-001 and 15872H-034) had higher aPTT and plasma concentration levels.

TABLE B
Effect of Formulations on aPTT (Multiple of Baseline)

| Sample | | | | |
|---|---|---|---|---|
| Time | | | | |
| (h) | 15860H-001 | 15872H-034 | 15872H-036 | 15872H-035 |
| 0.5 | 2.85 ± 1.05 | 1.10 ± 0.25 | 1.52 ± 0.46 | 1.10 ± 0.09 |
| 1 | 3.79 ± 0.98 | 1.20 ± 0.24 | 2.09 ± 0.61 | 1.64 ± 0.08 |
| 2 | 3.53 ± 0.70 | 3.07 ± 0.85 | 2.87 ± 1.09 | 3.26 ± 0.18 |
| 4 | 3.76 ± 0.66 | 3.03 ± 0.86 | 3.37 ± 0.78 | 3.65 ± 0.64 |
| 8 | 3.41 ± 0.66 | 1.85 ± 0.27 | 2.29 ± 0.70 | 2.33 ± 0.42 |
| 24 | 2.34 ± 0.59 | 1.08 ± 0.20 | 1.71 ± 0.75 | 1.51 ± 0.25 |
| 36 | 1.00 ± 0.06 | 0.72 ± 0.13 | 1.28 ± 0.29 | 1.35 ± 0.20 |
| 48 | 0.80 ± 0.12 | 0.85 ± 0.09 | 1.46 ± 0.52 | 1.18 ± 0.25 |

TABLE C
Mean Plasma Concentrations (ng/mL) Single Oral (200 mg) Dose

| Formulation | Subj | 0 hrs | 0.5 hrs | 1 hrs | 2 hrs | 4 hrs | 8 hrs | 24 hrs | 36 hrs | 48 hrs |
|---|---|---|---|---|---|---|---|---|---|---|
| 15860H-001 | 1 | 0.00 | 3770 | 1740 | 4200 | 2230 | 527 | 25.1 | 6.53 | 2.05 |
| | 2 | 0.00 | 146 | 405 | 1320 | 1190 | 397 | 17.2 | 3.54 | 0.00 |
| | 3 | 0.00 | 1500 | 8930 | 7480 | 5560 | 717 | 27.7 | 5.20 | 2.61 |
| | 4 | 0.00 | 2940 | 3300 | 4830 | 3940 | 795 | 23.6 | 2.75 | 0.00 |
| | Mean | 0.00 | 2089 | 3594 | 4458 | 3230 | 609 | 23.4 | 4.51 | 1.17 |
| | SD | 0.00 | 1599 | 3749 | 2529 | 1923 | 181 | 4.47 | 1.69 | 1.36 |
| 15872H-034 | 1 | 0.00 | 4.82 | 34.0 | 361 | 1230 | 636 | 19.5 | 2.96 | 0.00 |
| | 2 | 0.00 | 2.46 | 319 | 1890 | 1920 | 505 | 24.3 | 3.14 | 0.00 |
| | 3 | 0.00 | 155 | 261 | 1940 | 1160 | 390 | 29.0 | 6.31 | 2.46 |
| | 4 | 0.00 | 130 | 602 | 4170 | 4310 | 1310 | 31.5 | 5.47 | 2.62 |
| | Mean | 0.00 | 73.1 | 304 | 2090 | 2155 | 710 | 26.1 | 4.47 | 1.27 |
| | SD | 0.00 | 80.8 | 234 | 1568 | 1477 | 412 | 5.3 | 1.68 | 1.47 |
| 15872H-036 | 1 | 0.00 | 67.2 | 198 | 730 | 363 | 161 | 377 | 53.9 | 19.8 |
| | 2 | 0.00 | 110 | 342 | 972 | 2030 | 626 | 33.8 | 8.03 | 2.81 |
| | 3 | 0.00 | 108 | 292 | 211 | 71.6 | 83.3 | 23.2 | 4.64 | 0.00 |
| | 4 | 0.00 | 7.97 | 166 | 820 | 3610 | 792 | 33.8 | 6.35 | 2.42 |
| | Mean | 0.00 | 73.3 | 250 | 683 | 1519 | 416 | 117 | 18.2 | 6.26 |
| | SD | 0.00 | 47.8 | 81.6 | 330 | 1640 | 347 | 173 | 23.8 | 9.11 |
| 15872H-035 | 1 | 0.00 | 66.8 | 271 | 1170 | 431 | 255 | 115 | 23.4 | 9.87 |
| | 2 | 0.00 | 56.2 | 65.4 | 1410 | 2600 | 1650 | 92.2 | 27.5 | 10.7 |
| | 3 | 0.00 | 68.2 | 370 | 1920 | 3790 | 858 | 21.5 | 3.60 | 0.00 |
| | 4 | 0.00 | 18.7 | 429 | 3450 | 6690 | 1400 | 79.8 | 15.4 | 4.15 |
| | Mean | 0.00 | 52.5 | 284 | 1988 | 3378 | 1041 | 77.1 | 17.5 | 6.18 |
| | SD | 0.00 | 23.1 | 160 | 1024 | 2610 | 619 | 39.8 | 10.5 | 5.05 |

The slug capsules according to the present invention increased the aPTT (activated partial thromboplastin time) to the therapeutic range of 1.5 to 2.5 at the 24 hour time point. The activated partial thromboplastin time is a measure the blood coagulation time relative to the baseline.

While the foregoing specification teaches the principles of the present invention, with examples provided for the purpose of illustration, it will be understood that the practice of the invention encompasses all of the usual variations, adaptations and modifications as come within the scope of the following claims and their equivalents.

Throughout this application, various publications are cited. The disclosure of these publications is hereby incorporated by reference into this application to describe more fully the state of the art to which this invention pertains.

What is claimed is:

1. A monolayered gastro-retentive sustained release formulation retaining the dosage form in the stomach for a prolonged period.

2. The formulation of claim 1, further comprising a polymer selected from hydroxypropylmethylcellulose, hydroxypropylcellulose or polyvinylacrylate or mixtures thereof.

3. The formulation of claim 2, wherein the polymer is hydroxypropylmethylcellulose.

4. The formulation of claim 1, wherein the therapeutic agent is selected from the group consisting of therapeutic, chemotherapeutic, antibiotic antidiabetic, anti-cancers, anti-fungals, anti-filarial, antiviral.agents, lipid lowering agents, analgesics, non-steroidal anti-inflammatory agents, anti-ulcer agents, anti-epileptics, anti gout, immunosuppressants, drugs for respiratory therapy, dopaminergic agents, skeletal muscle relaxants, cardiovascular agents, thrombin inhibitors, antipsychotics or those drugs which does not show uniform absorption characteristic throughout the length of the gastrointestinal tract.

5. The formulation of claim 1, wherein the therapeutic agent is a compound of Formula (I):

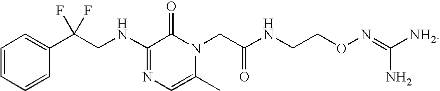

6. The formulation of claim 5, wherein the therapeutic agent is present in amount of about 200 mg.

7. The formulation of claim 5, wherein the formulation is in the form of a loosely-compacted slug.

8. Use of the formulation of claim 1 for treatment of disease conditions as described in any preceding claims above.

9. Use of the formulation of claim 5 for treatment of thrombosis.

10. A process for preparing a gastroretentive formulation comprising the step of admixing the formulation of claim 1 with one or more excipients.
